(19) United States
(12) Patent Application Publication
(10) Pub. No.: US 2008/0312615 A1
(43) Pub. Date: Dec. 18, 2008

(54) REMOVABLE SURGICAL DRAIN POCKET/POUCH

(76) Inventor: Carol Hunter, Merrick, NY (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 12/139,399

(22) Filed: Jun. 13, 2008

Related U.S. Application Data
(60) Provisional application No. 60/934,386, filed on Jun. 14, 2007.

(51) Int. Cl. A61F 5/44; A41D 10/00; A41D 13/12

(52) U.S. Cl. 604/345; 2/114

(57) ABSTRACT

A support device and method for carrying drainage receptacles connected to a post-operative surgery patient user. A removable pocket has a front panel and a back panel, wherein said back panel defines a first fastener strip that is removably attached to a second fastener strip located on a garment, and wherein placement of said second fastener strip can be pre-determined or determined by the user.

### CROSS-REFERENCE TO RELATED APPLICATIONS

This application claims the benefit of U.S. provisional application Ser. No. 60/934,386, entitled “Removable Surgical Drain Pocket/Pouch,” filed Jun. 14, 2007, which is incorporated by reference herein in its entirety.

### BACKGROUND

Major surgeries such as lumpectomies and mastectomies are traumatic to the human body. In some cases, the removal of tissue creates a body cavity and the remaining lymph nodes may secrete fluid that collects in the cavity. To relieve swelling and infection caused by excess fluid in the body, a drainage tube may be inserted through the surgical incision into the cavity. Such drain tubes have perforated ends through which fluid enters the tube from the cavity. The drainage tube that is sutured into the body of the patient generally hangs downwardly and ends with a collection receptacle. The receptacle is a container that collects excess fluid from the body and is generally in the shape of a bulb but can vary in its size and shape. The number of the drainage tubes and their respective receptacles may vary depending on the surgery and the amount of fluid buildup expected.

Such a configuration can cause both physical and emotional discomfort, as the drainage tubes and receptacles can be cumbersome, painful and/or embarrassing for the user. Managing the tubes and monitoring fluid collection in their respective receptacles can be awkward and unwieldy in that the receptacles need to be stored and supported without tangling the tubes and interfering with the user's clothing and movements. Thus, it is desirable to design a support system that makes the drainage receptacles easily accessible for removing and draining when necessary while also supporting the receptacle, preventing the receptacle from tugging on the drainage tubes.

Because the number of drainage tubes and the shapes of their receptacles can vary, it is desirable to be able to customize the positioning of the support system in order to accommodate the different types of configurations.

It is also desirable to design a support system that can be concealed under regular clothing so that the user's normal appearance is not affected. A common concern for a woman who undergoes a mastectomy is having to learn to cope with an altered body image as she recovers from the surgery. During this emotional time, such post-operative patients have a psychological need to feel and look as normal as possible.

### SUMMARY

The devices, systems and methods described herein are done so by way of exemplary embodiments. These embodiments are discrete examples only and in no way should be interpreted as limiting the inventions. The devices, systems and methods described herein address a system and method for carrying and concealing post surgical drainage containers/receptacles that are used to collect bodily fluids after certain types of surgery.

In the above-referenced devices, systems and methods, one or more removable pockets can be used to carry and conceal post surgical drain bulbs/receptacles. In one embodiment, a removable pocket is designed for each pocket to accommodate up to two drain receptacles as the number and shape of the receptacles may vary. The pockets can be easily attached to and detached from garments such as clothing and other surfaces. The pocket can be made of any type of fabric including waterproof fabric or plastic. It can be disposable or reusable. It can be attached by a hook and loop fastener, such as those commonly sold under the trademark “Velcro,” either by iron-on, sewn-on, or sticky-type removable fasteners. Buttons, zippers, snaps, hooks or any other suitable attachment can be used.

According to one exemplary embodiment, the pocket can contain a stretch opening made of elastic or rubber material that will open when pulled and spring back and close by itself, which allows easy access to drain receptacles. This pocket/pouch allows for the receptacles to be held and hidden unobtrusively inside the patients clothing, which allows for patient comfort and can enhance the psychological well being of the patient.

In another exemplary embodiment, the removable pocket can be attached to a garment by the use of two complementary mating fastener strips. The first fastener strip can be sewn on or otherwise attached to the pocket. The second fastener strip can be sewn on or otherwise attached to the garment itself. The removable pouch/pocket can then be removably attached to one's garment via the complementary fasteners on the two fastener strips.

### BRIEF DESCRIPTION OF THE DRAWINGS

Features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 is a front view of a pouch/pocket according to one embodiment of the invention.

FIG. 2 is a back view of a pouch/pocket according to one embodiment of the invention.

FIG. 3 shows a complimentary hook and loop fastener strip that the pouch/pocket attaches to.

FIG. 4 is a front view of a pouch/pocket with the drain receptacles inserted according to one embodiment of the invention.

FIG. 5 is a frontal perspective view of a pouch/pocket according to one embodiment of the invention.

FIGS. 6A and 6B show a front view of a garment being turned inside-out with a first configuration of fastener strips, with and without removably attached pockets.

FIGS. 7A and 7B show a front view of a garment being turned inside-out with a second configuration of fastener strips, with and without removably attached pockets.

FIG. 8 is a front view of a jacket with pouches/pockets concealed inside.

FIG. 9 is a schematic view of the inside of a jacket with pouches/pockets removably attached to fastener strips.

### DETAILED DESCRIPTION OF A PREFERRED EMBODIMENT

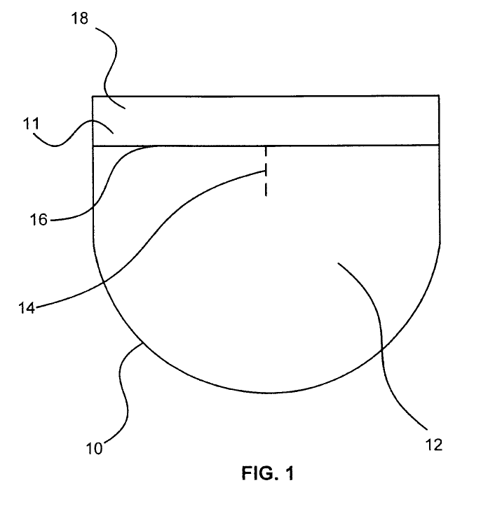

Referring now in detail to the drawings, an embodiment of the present invention shown in FIGS. 1-5 is a removable pocket/pouch 10 that can be customized in size and shape and affixed to clothing to carry and conceal post surgical drain bulbs/receptacles.

The front view of one embodiment of the invention is shown in FIG. 1 disclosing a pocket 10 having an open upper end 11 and preferably defines a front panel 12 and a back panel 18. It is understood that the pocket 10 can be formed of fabric such as canvas, leather, plastic, cloth, or any other suitable material. Pocket 10 can be disposable or reusable.

According to one embodiment, the pocket 10 can be divided into two parts at the top edge 16 of the front panel 12. The division 14 can be sewn, glued, taped, attached, or otherwise created using any method of dividing the pocket 10 to separately house more than one drain bulb/receptacle. The pocket 10 can be divided or it can remain undivided. The top edge 16 of the front panel 12 forms an open upper end 11 with the back panel 18. When the user opens the pocket, part of the front panel 12 is pulled away from the back panel 18, such that a pocket mouth is created at the upper end 11 and the pocket interior is fully exposed, providing easy access for placing the drainage receptacle within the pouch interior. The top edge 16 may be made of or lined with elastic or rubber material such that it can spring back and close by itself after opening.

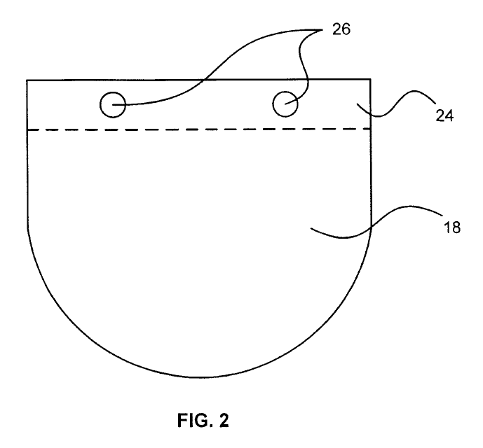

FIG. 2 shows a back view of the pocket 10. In the embodiment illustrated in FIG. 2, one or more fasteners or securement members 26 is provided on the back panel 18. The fasteners 26 can be attached either directly to the back panel 18 or to a first fastener strip 24 that is attached to the back panel 18. Alternatively, the fastener strip 24 itself may act as a fastener by defining a mating surface comprising a fabric hook and loop fastener (i.e., either the hook fastener strip side or the loop fastener strip side) such as those commonly sold under the trademark “Velcro.” In an exemplary embodiment, the non-mating surface of the first fastener strip 24 is affixed along the upper border of the back panel 18 of the pocket 10.

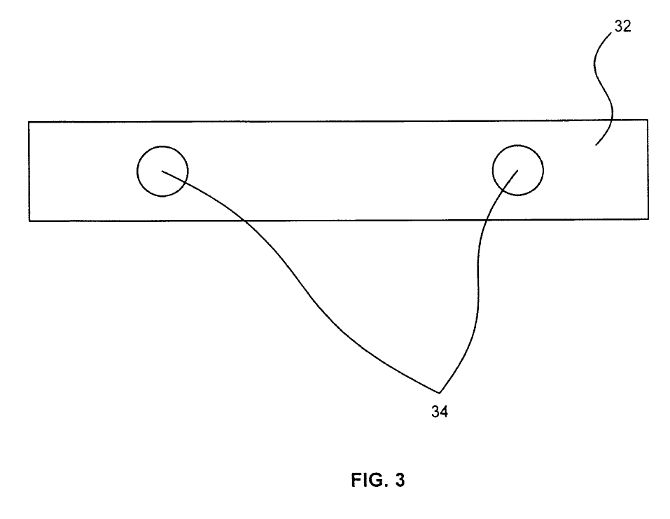

To secure the removable pocket 10 to the user patient's clothing, a second fastener strip 32 containing one or more complementary mating fasteners 34 is provided, as illustrated in FIG. 3. The second fastener strip 32 can be attached to the user's clothing and positioned in accordance with the user's convenience and preference. It can be sewn on, ironed on or attached to the inside of the user's clothing using sticky back. Once the fastener strip 32 is in place, then the fasteners 34 attached thereto can be paired with their complementary fasteners 26 attached to the pocket 10 so as to releasably secure the pocket 10 to the user's clothing.

In an alternative embodiment, the fasteners 34 can be sewn on or otherwise attached directly to the user's clothing. Under this configuration, the fasteners 34 can be releasably secured to the fasteners 26 on the pocket 10 without the use of an additional fastener strip. For example, fasteners 34 may comprise buttons sewn onto the inside of a piece of clothing, while fasteners 26 may comprise complementary fasteners attached to the pocket 10 or simply openings on the pocket 10 that would mate with the buttons and releasably secure the pocket 10 to the buttons.

According to one exemplary embodiment, the first fastener strip 24 and second fastener strip 32 contain complementary sides of a fabric hook and loop fastener. In this instance, the pouch/pocket 10 can be removably attached to one's garment many times just by pressing opposing hook and loop fastener strips together. It is understood that the hook and loop fastener strips can be readily interchanged such that fastener strip surface 24 comprises hooks while fastener strip surface 32 comprises loops and vice versa, within the scope of this invention and its associated claims. It is also understood that while this disclosure refers to and illustrates at times, hook and loop fasteners, that in fact fasteners or securement members 26 and 34 used in connection with this invention are more broadly defined, including but not limited to hook and loop fasteners, adhesive strips, flexible plastic tabs, snaps, magnets, buttons, or any other attachment means that would achieve the desired securement.

The pouch/pocket, having the first fastener strip 24 on the back panel, can be releasably secured to the second fastener strip 32, the placement of which can be customized according to the user's preference. Depending on the style of clothing worn by the user, the fastener strip 32 can be placed strategically at a location inside the garment so as accommodate one or more pockets to conceal the receptacles being carried underneath the garment. For example, in an outerwear such as a sweatshirt or a jacket, where the mid-section is normally loose-fitting, the fastener strip 32 might be positioned near the abdomen so that the bottom of the pocket lines up above the waist. Similarly, when the user has more than one tube and receptacle to carry, he or she can choose how to position the multiple pockets/pouches inside a piece of clothing. In this way the drainage receptacles are hidden and the aesthetic appearance of the garment is unchanged.

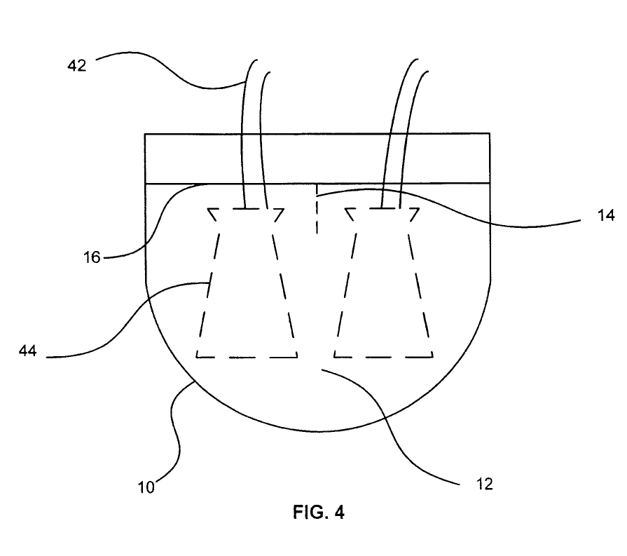

As shown in FIG. 4, the removable pocket/pouch 10 is designed for carrying post-surgical drainage tubes 42 and receptacles 44 in a suspended position thereby removing tension along tube 42, preventing tube 42 from pulling on the patient. It is also advantageous to be able to customize the placement of the pockets 10 to accommodate and conceal drainage tubes and receptacles originating from different parts of the body. In addition, the pockets may be placed and configured for easy accessibility. This versatility ensures that any drainage receptacle with any surgery may be easily carried and monitored.

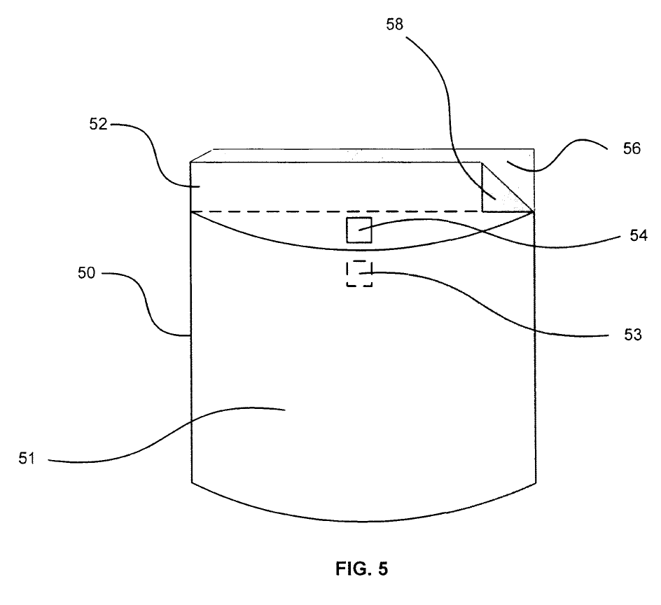

Referring to FIG. 5, another exemplary embodiment of a removable pouch/pocket 50 is shown. According to this embodiment, the pocket 50 having a pocket interior is defined by a front panel 51 and a back panel 52. Complementary fabric hook and loop fasteners 53 and 54 are positioned on the front panel 51 and back panel 52, respectively, and are used to secure the top of the front panel 51 to the back panel 52 and to divide the pocket into two compartments. Pursuant to the division, a drainage receptacle can be placed in each compartment and the receptacles can sit in the pocket in an upright position to prevent accidental spillage. The upper end of the back panel facing away from the front panel defines a first fastening strip 58, which can be a hook and loop fastener strip that is removably attached to a second fastening strip 56. The user can position the pocket 50 anywhere on a garment by sewing or otherwise affixing the second fastening strip 56 to a preferred location on the garment. In conjunction with the second fastening strip 56, the user can use the pocket 50 to support the drainage tubes and receptacles under the garment.

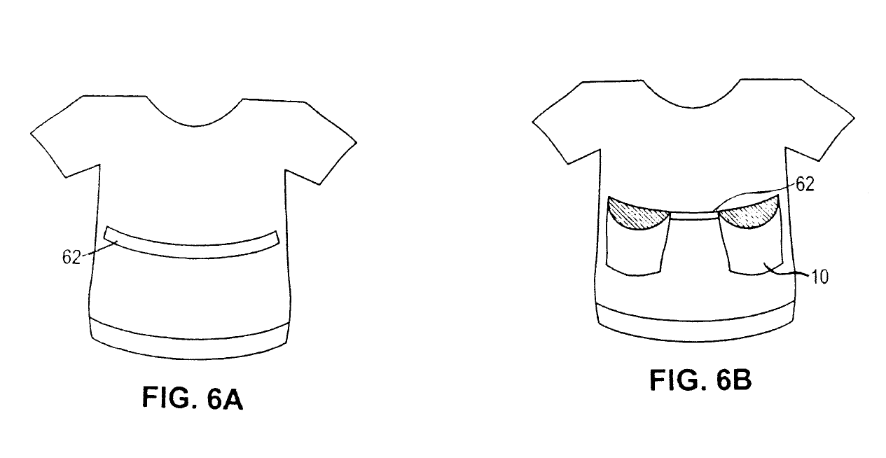

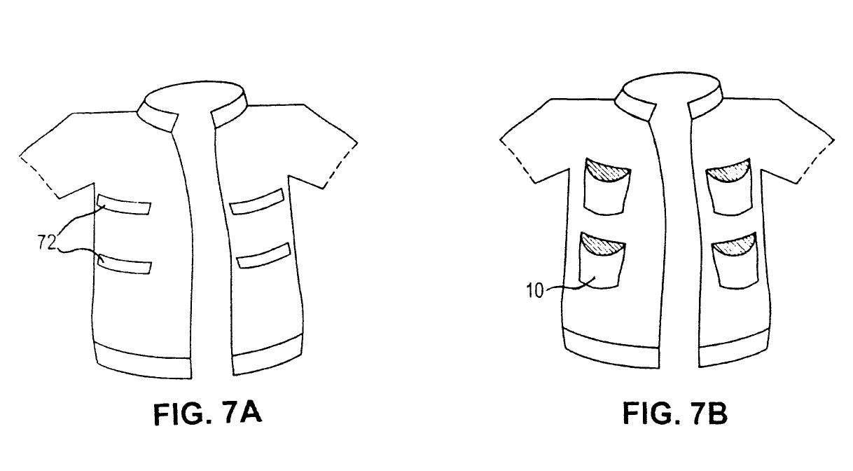

According to another aspect of the invention, a garment may contain one or more fastener strips that are already pre-sewn or otherwise affixed to the garment, so as to accommodate removable pockets with complementary fastener strips. As illustrated in FIGS. 6A and 7A, one or more fastener strips of various lengths can be affixed to predetermined locations on the inside of a garment. The garment, for example, can be a shirt or some type of outerwear, such as a coat, sweater, sweatshirt or jacket. The length of the fastener affixed to the garment does not have to be of equal length to that of the complementary fastener on the pocket. As shown in FIGS. 6A and 7A, the fastener affixed to the garment can span the inside of the garment as one long strip 62 or several shorter strips 72 to accommodate multiple pockets with complementary fasteners. As shown in FIGS. 6B and 7B, one or more pockets 10 is adapted to releasably attach to the fastener strip(s).

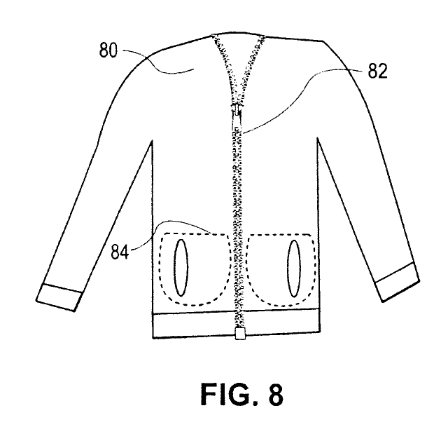

The placement of the pocket/pouch allows the user to hold and hide one or more drainage receptacles unobtrusively under the garment. The embodiment of the invention depicted in FIG. 8 shows a jacket 80 capable of carrying one or more pockets 84 that are concealed from public view. According to one variation, the jacket 80 defines an opening 82 in the front closeable by zippers or buttons. This configuration makes wearing the jacket easier than a garment with no front opening, such as a t-shirt, because the user would not have to raise his or her arms in order to wear the jacket. It also facilitates monitoring and draining the receptacles because the user would only have to unzip or unbutton the jacket to reach the receptacles, rather than having to remove the entire garment.

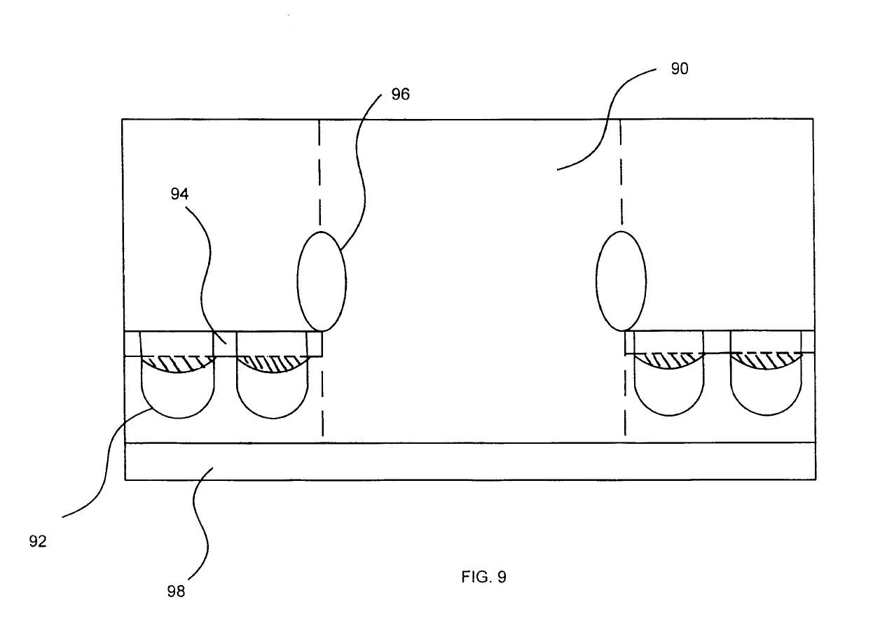

In a variation of the present invention illustrated in FIG. 9, fastener strips 94 can be placed on each side of the inside torso portion 90 of a garment near the mid-section. This configuration enables the pockets to be removably attached at the mid-section above the waistband 98, where there can be enough space to conceal the pockets without showing a bulge in the garment. The garment is also adapted to have multiple pouches/pockets 92 on each side to accommodate multiple receptacles. The torso portion 90 contains sleeve openings 96, so that the garment can include sleeves or it can be sleeveless. According to one embodiment, the garment is loose-fitting in the mid-section area between the fastener strip 94 and the waistband 98 such that any pockets 92 would be easily concealed under the garment.

While the invention is susceptible to various modifications and alternative forms, specific examples thereof have been shown in the drawings and are herein described in detail. It should be understood, however, that the invention is not to be limited to the particular form disclosed, but to the contrary, the invention is to cover all modifications, equivalents, and alternatives falling within the spirit of the appended claims.

What is claimed is:

1. A post-surgical drainage support system for carrying post-surgical drainage tubes and receptacles, said support system comprising:
a drainage tube connectable to a user for draining bodily fluids away from said user;
a drainage receptacle connecting to said drainage tube for receiving bodily fluids; and
a pocket having a front panel and a back panel defining a pocket interior for receiving and supporting said drainage tube and said drainage receptacle, said back panel comprising a first fastener strip that is removably attachable to a second fastener strip affixable to a garment.

2. The support system of claim 1 wherein said first fastener strip defines a first plurality of fasteners that are complementary to a second plurality of fasteners on said second fastener strip.

3. The support system of claim 1 wherein placement of said second fastener strip is determined by said user.

4. The support system of claim 1 wherein said garment is an outerwear.

5. The support system of claim 1 wherein said pocket is divided into at least two compartments.

6. A garment for carrying drainage receptacles connected to a post-operative surgery patient user, comprising:
a torso portion, having an inside and an outside;
a pocket having a pocket interior for receiving and supporting a drainage tube and a drainage receptacle; and
a first fastener strip having a first side that is affixed to said pocket and a second side adapted to be removably attached to a second fastener strip affixed to said inside of said torso portion.

7. The garment of claim 6, further comprising a plurality of pockets having fasteners that is removably attached to said second fastener strip.

8. The garment of claim 6, further comprising a plurality of fastener strips affixed on said torso portion to accommodate a plurality of pockets.

9. A method to carry post-surgical drainage receptacles, comprising:
attaching a fastener strip to a piece of clothing of a user's choosing at a location of said user's choosing;
securing a pocket to said fastener strip; and
suspending a drainage receptacle in said pocket.

10. The method of claim 9, wherein said pocket defines a plurality of fasteners adapted to be releasably attached to a plurality of complementary fasteners on said fastener strip.
